(19) United States
(12) Patent Application Publication
(10) Pub. No.: US 2025/0157600 A1
(43) Pub. Date: May 15, 2025

(54) PATHOGEN DETECTION AND IDENTIFICATION SYSTEM AND METHOD

(71) Applicant: THE JOHNS HOPKINS UNIVERSITY, Baltimore, MD (US)

(72) Inventors: Debadrita PARIA, Baltimore, MD (US); Arijit GHOSH, Baltimore, MD (US); David H. GRACIAS, Baltimore, MD (US); Ishan BARMAN, Baltimore, MD (US)

(21) Appl. No.: 18/838,790

(22) PCT Filed: Feb. 27, 2023
(86) PCT No.: PCT/US2023/013934

§ 371 Date: Aug. 15, 2024

Related U.S. Application Data
(60) Provisional application No. 63/314,892, filed on Feb. 28, 2022.

(51) Int. Cl. G16H 10/40; G01N 21/65; G01N 33/543; G16H 40/63

(52) U.S. Cl. CPC G16H 10/40; G01N 21/658; G01N 33/54373; G16H 40/63

(57) ABSTRACT

A pathogen detection system and method are disclosed. The pathogen detection system includes a pathogen sensor comprising a plasmonically active pathogen sensing area that comprises a hybrid structure of electrically conductive surface and metal nanofractals, forming a surface enhanced Raman spectroscopic (SERS) substrate and a controller comprising a memory storing one or more trained machine learning algorithms, that perform label-free identification and differentiation of a pathogen or the pathogen and a pathogen mutation based on acquired Raman spectra.

### CROSS-REFERENCE TO RELATED APPLICATIONS

This application is the national stage entry of International Patent Application No. PCT/US2023/013934, filed on Feb. 27, 2023, and published as WO 2023/164207 A1 on Aug. 31, 2023, which claims the benefit of U.S. Provisional Patent Application No. 63/314,892, filed on Feb. 28, 2022, which are hereby incorporated by reference in their entireties.

### GOVERNMENT FUNDING

This invention was made with government support provided by the National Science Foundation under grant no. CMMI2033349. The Government has certain rights in the invention.

### FIELD

This invention relates generally to pathogen detection and identification, and more particularly, to a low-cost sensing system and method for pathogen detection and identification using fractal nanostructures.

### BACKGROUND

Coronavirus disease, which causes moderate to severe respiratory illness, was first detected in Wuhan, China in 2019 and is the cause of the ongoing pandemic. It has resulted in over 4.6 million deaths till date. Slow vaccination rates in many countries make mass testing an absolute necessity for easing travel restrictions and restarting the economy. But the existing testing methods are either expensive, time consuming or often suffer from false positive. Moreover, they require operator expertise and elaborate reagents which make scaling-up the testing method impractical. Thus, there is an urgent need for an inexpensive mass testing method that can produce an accurate result in a matter of minutes, requiring minimal technical expertise.

### SUMMARY

According to examples of the present disclosure, a pathogen detection system is disclosed. The pathogen detection system comprises a pathogen sensor comprising a plasmonically active pathogen sensing area that comprises a hybrid structure of electrically conductive surface and metal nanofractals, forming a surface enhanced Raman spectroscopic (SERS) substrate and a controller comprising a memory storing one or more trained machine learning algorithms, that perform label-free identification and differentiation of a pathogen or the pathogen and a pathogen mutation based on acquired Raman spectra.

According to examples of the present disclosure, a method for label-free detection to identify and differentiate various pathogens and their mutations based on applying a machine learning algorithm on surface enhanced Raman spectra acquired on a nano fractal substrate is disclosed. The method comprises depositing a pathogen specimen onto a pathogen sensor comprising plasmonically active noble metal nanostructures; illuminating the pathogen specimen that is deposited with a laser; acquiring surface enhanced Raman spectroscopic (SERS) signal from the pathogen specimen; and determining and identifying one or more pathogens and their mutations by applying the machine learning algorithm on the SERS signal that is acquired.

According to examples of the present disclosure, a method of forming a pathogen sensor is disclosed. The method comprises forming a first graphene or a 2D material layer on a top surface of a substrate; and forming a first metallic fractal nanostructure on a top surface of the first graphene or a 2D material layer.

Various additional features can be included in the method of forming the pathogen sensor including one or more of the following features. The method of forming a pathogen sensor of claim 18, further comprising forming a second graphene or a 2D material layer on the top surface of the first metallic fractal nanostructure and forming a second metallic fractal nanostructure on a top surface of the second graphene or a 2D material layer. The method of forming a pathogen sensor further comprises forming a second graphene or a 2D material layer on the top surface of the first metallic fractal nanostructure and forming of layer of a plurality of metallic nanodots on a top surface of the second graphene or a 2D material layer. The substrate comprises a flat transparent material or a flat opaque material, the flat transparent material or a flat opaque material comprising silicon, germanium, glass, quartz, polymer, or a gel. The substrate comprises a flexible material or a rigid material, wherein the flexible material comprises, a Kapton tape, a polyimide film, a polymer, a gel, or a polydimethylsiloxane (PDMS).

According to examples of the present disclosure, a method of forming a pathogen sensor is disclosed. The method comprises forming an electrically conductive layer formed on a top surface of a substrate; and forming a metallic fractal nanostructure formed on a top surface of the electrically conductive layer.

Various additional features can be included in the method of forming the pathogen sensor including one or more of the following features. The substrate comprises a flat transparent material or a flat opaque material, the flat transparent material or a flat opaque material comprising silicon, germanium, glass, quartz, polymer, or a gel. The substrate comprises a flexible material or a rigid material, wherein the flexible material comprises a polyimide film, a polymer, a gel, or a polydimethylsiloxane (PDMS).

Various additional features can be included in the pathogen detection system, pathogen sensor, and/or pathogen detection methods, including one or more of the following features. The controller is further configured to determine one or more of the following based on the one or more trained machine learning algorithms and the acquired Raman spectra: pathogen concentrations, or differences in various protein and genomic compositions of the pathogen or the pathogen and the pathogen mutation. The pathogen detection system further comprises a bench top Raman spectrometer, a hand-held Raman spectrometer, or a portable Raman spectrometer. The plasmonically active pathogen sensing area is fabricated using metal nano fractals on an electrically conducting surface, through a process of electrodeposition. The metal nano fractals comprise silver, gold, platinum, copper, aluminum, or any combination/alloy of these metals. The electrically conducting surface is a 2D material, wherein the 2D material comprises graphene, MoS₂, black phosphorus, WS₂ or other 2D electrically conductive materials. The 2D material is modified or unmodified, wherein the modified comprises doping, functionalization, oxidation, or patterning of the electrically conductive surface. The SERS substrate comprises a metal coated silicon wafer, a metal coated glass wafer, or a metal coated quartz wafer. The electrically conductive surface for the SERS substrate comprises a metal, a conducting oxide, a doped semiconductor, or a conducting polymer, wherein the metal comprises gold, silver, platinum, or copper, the conducting oxide comprises indium tin oxide (ITO), zinc oxide, gallium oxide, indium oxide or tin oxide. The plasmonically active pathogen sensing area comprises a multilayered architecture in the form of graphene-nanofractals-graphene-nanofractals fabricated by consecutive graphene transfers and electrodeposition. The plasmonically active pathogen sensing area comprises a multilayered architecture in the form of graphene-nanofractals-graphene-nanodots fabricated by consecutive graphene transfers and electroplating, physical vapor deposition, chemical synthesis, or combinations thereof. The plasmonically active pathogen sensing area comprises a substrate, a first graphene or other 2D material layer formed on a top surface of the substrate, and a first metallic fractal nanostructure formed on a top surface of the first graphene or other 2D material layer. The plasmonically active pathogen sensing area further comprises a second graphene or other 2D material layer formed on the top surface of the first metallic fractal nanostructure and a second metallic fractal nanostructure formed on a top surface of the second graphene or other 2D material layer. The plasmonically active pathogen sensing area further comprises a second graphene or other 2D material layer formed on the top surface of the first metallic fractal nanostructure and a plurality of metallic nanodots formed on a top surface of the second graphene or other 2D material layer. The SERS substrate comprises a flat transparent material or a flat opaque material, the flat transparent material or a flat opaque material comprising silicon, germanium, glass, quartz, polymer, or a gel. The SERS substrate comprises a flexible material or a rigid material, wherein the flexible material comprises a Kapton tape, a polyimide film, a polymer film, a gel film, or a polydimethylsiloxane film (PDMS). The SERS substrate has a dimension ranging from about a micron to about a meter. The metal nanofractals have a dimension ranging from about a sub-nanometer to about a millimeter. The pathogen sensor is applied to a micro or macro substrate, wherein the micro or macro substrate comprises a flute that operates as a breath sensor, a swab, a pin, a needle, a cotton swab, a fabric, a surface of a furniture, a surface of a wall, or a skin tattoo. The pathogen sensor is arranged on a period pattern, a microwell, or other kinds of regular or irregular patterns. The electrically conductive surface is patterned to enable deposition of the metal nanofractals at specific locations. A density or a coverage of the metal nanofractals is controlled by using different current densities ranging from about an picoampere per square meter to about an ampere per square meter, different electrolyte concentration and time of deposition ranging from about a few milliseconds to about a few hours, or both. The metal nanofractals are modified chemically with DNA or biomolecules for improved sensitivity and multiplexed detection, wherein the biomolecules comprise an antibody or an antigen. The metal nanofractals comprise fluorophores or Raman reporters that are sprinkled or functionalized to improve sensitivity or added functionality. The pathogen sensor is a stand-alone device, combined with a microfluidic device, or combined with a lab-on-chip device for easier sample collection and analysis along with multiplexed detection. The pathogen comprises a virus, a bacterium, a biomolecule, or a protein. The virus comprises influenza, Zika, SARS-CoV-2, a coronavirus, Marburg, or other respiratory or non-respiratory viruses. The label-free identification and differentiation comprises distinguishing between RNA of wild type and mutated SARS-CoV-2 virus or other viruses/pathogens. The label-free identification and differentiation is based on a specimen acquired by saliva, a swab, blood plasma, urine, tears, or any form of physiological body fluids carrying live or dead pathogen. The specimen is collected with or without further processing comprising a chemical or physical treatment. The specimen is collected by directly drop casting, placing the specimen on the pathogen sensor, blowing, sneezing, or coughing directly on the pathogen sensor. The acquired Raman spectra is preprocessed to correct for background conditions or to perform a normalization process. The one or more machine-learning algorithms are trained by dividing collected datasets into a training dataset, a validation dataset, and a test dataset. The one or more machine-leaning algorithms comprise an unsupervised classification algorithm that performs a data visualization operation and a feature extraction operation, wherein the unsupervised classification algorithm comprises principal component analysis, K-means clustering, or other visualization algorithms. The one or more machine-learning algorithms comprise a partial least squares regression analysis with or without additional outliner detection algorithm applied to an acquired pathogen dataset on the SERS substrate for determining of pathogen concentration of at least 10 copies/ml or higher. The additional outliner detection algorithm comprises a robust principal component analysis (PCA). The one or more machine-learning algorithms comprise one or more supervised machine learning algorithms, wherein the one or more supervised machine learning algorithms comprise a logistic regression, a support vector machine, a decision tree, a random forest that is used to identify one or more types of pathogens in a pathogen specimen and to differentiate between various mutations. The one or more supervised machine learning algorithms are used to identify different pathogen compositions, wherein the pathogen compositions comprise proteins, lipids, or genomic contents.

According to examples of the present disclosure, the detection system can be integrated with one or more additional systems including, but are not limited to, a saliva/blood sample collection unit, a microfluidic system, or a lateral flow assay.

Advantages of the embodiments will be set forth in part in the description which follows, and in part will be understood from the description, or may be learned by practice of the invention. The advantages will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate embodiments of the invention and together with the description, serve to explain the principles of the invention.

### BRIEF DESCRIPTION OF THE FIGURES

FIG. 1 shows a pathogen detection structure and method according to examples of the present disclosure.

FIG. 2A, FIG. 2B, FIG. 2C, and FIG. 2D shows various types of sensor architectures according to examples of the present disclosure.

FIG. 3A shows a photograph of a sensor architecture with silver fractal nanostructures electroplated on a single layer graphene with a SiO₂—Si layer as substrate base according to examples of the present disclosure.

FIG. 3B and FIG. 3C show closeup and large area scanning electron microscope views, respectively, of the silver fractal nanostructures on graphene according to examples of the present disclosure.

FIG. 4A shows a scanning electron microscope view of the silver fractal nanostructures on gold coated Si according to examples of the present disclosure.

FIG. 4B shows a photograph of the example device in the inset of FIG. 4A.

FIG. 5 shows a scanning electron microscope view of the silver fractal nanostructures electrodeposited on ITO coated glass surface with various parameters marked in the figure according to examples of the present disclosure.

FIG. 6 shows a Raman spectra of cell lysates containing inactivated SARS-CoV-2 acquired on silver fractal nanostructures after post-processing according to examples of the present disclosure.

FIG. 7 shows a principal component analysis that show spectral data points belonging to RNA extracted from wild and mutated SARS-CoV-2 cluster together according to examples of the present disclosure.

FIG. 8 shows a predicted concentration versus true concentration of inactivated SARS-CoV-2 in cell lysates by partial least square regression according to examples of the present disclosure.

FIG. 9 shows a computer system according to examples of the present disclosure.

### DETAILED DESCRIPTION

Reference will now be made in detail to the present embodiments, examples of which are illustrated in the accompanying drawings. Wherever possible, the same reference numbers will be used throughout the drawings to refer to the same or like parts.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 5. In certain cases, the numerical values as stated for the parameter can take on negative values. In this case, the example value of range stated as “less that 10” can assume negative values, e.g. −1, −2, −3, −10, −20, −30, etc.

The following embodiments are described for illustrative purposes only with reference to the Figures. Those of skill in the art will appreciate that the following description is exemplary in nature, and that various modifications to the parameters set forth herein could be made without departing from the scope of the present invention. It is intended that the specification and examples be considered as examples only. The various embodiments are not necessarily mutually exclusive, as some embodiments can be combined with one or more other embodiments to form new embodiments.

Generally speaking, examples of the present disclosure provide for a low-cost label-free rapid sensing device for detection and classification of pathogens, including but are not limited to a virus, a bacterium, a biomolecule, or a protein. For example, the virus comprises influenza, Zika, SARS-CoV-2, a coronavirus, Marburg, or other respiratory or non-respiratory viruses. The label-free identification and differentiation can also distinguish between RNA of wild type and mutated SARS-CoV-2 virus or other viruses/pathogens. The sensing device uses a sensor, which is fabricated in a variety of rigid, flexible, micro and macro substrates. Combining label-free non-destructive Surface Enhanced Raman Spectroscopy (SERS) and machine learning (ML) with such a sensor fabric will enable highly sensitive detection of pathogens. In one non-limiting example, the sensor comprises a large area fractal nanostructures fabricated by electrochemical deposition in the order of seconds, on silver/gold, single layer graphene or other conducting flexible or rigid surfaces. SERS signal acquired from pathogen samples collected on the substrate presents unique vibrational fingerprints of the pathogen under investigation. Application of ML classifiers to the acquired raw spectra further enhances the sensitivity, specificity, and accuracy. Further integration of the fractal nanostructure with a microfluidic device will enable commercial grade collection and sensing with a portable Raman spectrometer for mass on-spot testing and screening at airports, shopping malls and hospitals.

In one non-limiting example, the present pathogen detection system uses a low-cost label-free sensing platform for ultrasensitive, fast, and accurate identification of pathogens, such as SARS-CoV-2 and other pathogens, by SERS acquired on silver nano-fractals on graphene/other conducting flexible substrates, that can be mass produced. Advanced machine learning algorithms are applied in the postprocessing stage for pathogen identification and classification of various pathogen, such as virus strains, with high degree of sensitivity and specificity.

Raman spectroscopic signature of pathogens are inherently weak. Thus, plasmonically active noble metal nanostructures are used to enhance the signal quality. Several techniques like chemical synthesis, lithography and physical vapor deposition can be used to fabricate plasmonic nanostructures in the past, but they are often expensive, time consuming and require sophisticated instruments and controlled manufacturing environments like a clean room. The present fabrication method involves a simple technique—electrochemical deposition for the fabrication of large area silver/gold fractal nanostructures on graphene and other conducting substrates. Such a facile technique enables low-cost mass-scale commercial production of such substrates for population wide testing of pathogen, such as SARS-CoV-2 and other viruses/pathogens.

Further integration of ML algorithms with the acquired Raman spectra on the substrates enables identification and differentiation various pathogens, such as various viruses and their mutations, as well as determine their pathogen or viral concentrations. ML algorithms can identify the subtle differences in the various protein and genomic compositions of the pathogens without the requirement of any additional sample processing or specialized technical expertise. As an example, use of ML can distinguish between RNA of wild type and mutated SARS-CoV-2 virus, which cannot be directly derived from the raw spectra. Thus, integration of ML algorithms with the present system and/or sensor can achieve a higher degree of specificity.

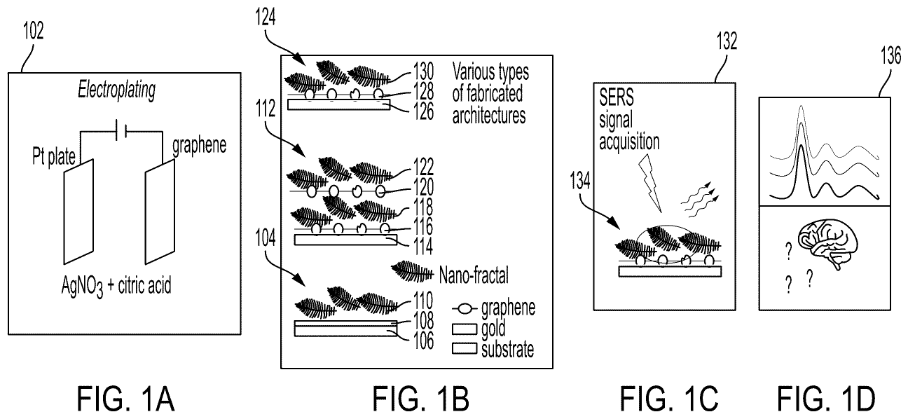

FIG. 1A, FIG. 1B, FIG. 1C, and FIG. 1D show a pathogen detection structure and method according to examples of the present disclosure. The pathogen detection structure is formed by fabricating silver nano fractals on graphene/other conducting surfaces using electroplating. This is followed by directly drop casting the pathogen sample on the substrate or integrating the substrate with a microfluidic collection system. SERS signal is collected by a Raman spectrometer or a portable hand-held Raman device. The raw spectra collected are postprocessed and various ML algorithms are applied for virus identification and classification.

As shown in FIG. 1A, the electroplating process 102 comprises passing an electrical current through a AgNO₃ plus citric acid solution where an electrically conductive material, such as platinum, from a platinum plate is deposited on a graphene substrate. Other electrically conductive materials, such as but not limited to, silver, gold, copper, aluminum, or any combination/alloy of these metals can be used. The electroplating process 102 creates metallic fractal nanostructures on the graphene or other conducting surfaces.

FIG. 1B shows various sensor architectures that can be fabricated using the electroplating process 102. A first sensor architecture 104 comprises a substrate layer 106, a gold layer 108 formed on a top surface of the substrate layer 106, and a nano-fractal layer 110 formed on a top surface of the gold layer 108. A second sensor architecture 112 comprises a substrate layer 114, a first graphene layer 116 formed on a top surface of the substrate layer 114, a first nano-fractal layer 118 formed on a top surface of the first graphene layer 116, a second graphene layer 120 formed on a top surface of the first nano-fractal layer 118, and a second nano-fractal layer 122 formed on a top surface of the second graphene layer 120. A third sensor architecture 124 comprises a substrate layer 126, a first graphene layer 128 formed on a top surface of the substrate layer 126, and a nano-fractal layer 130 formed on a top surface of the first graphene layer 128.

FIG. 1C shows a SERS signal collection process 132 that comprises placing a pathogen sample on the nano-fractal surface 134. FIG. 1D shows a postprocessing process 136 of the raw spectra followed by application of machine learning for increased sensitivity.

The sensor area of the sensor architectures is fabricated by electroplating, graphene transfer, and a single step physical vapor deposition. The fractal nanostructures formed have narrow branches and plasmonic gaps that enhances the electromagnetic near field allowing the amplification of SERS signal.

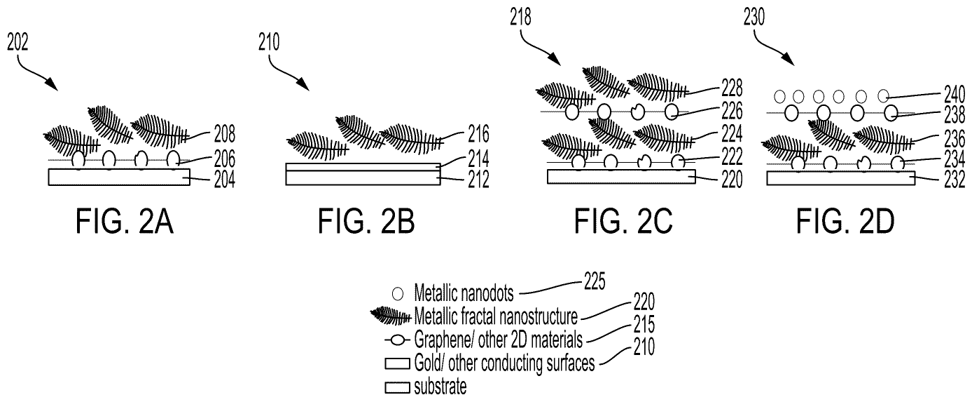

FIG. 2A, FIG. 2B, FIG. 2C, and FIG. 2D shows various types of sensor architectures according to examples of the present disclosure. FIG. 2A shows a first sensor architecture 202 that comprises a substrate layer 204, a graphene or other 2D material layer 206 formed on a top surface of the substrate layer 204, and a metallic fractal nano-fractal nanostructure layer 208 formed on a top surface of the graphene or other 2D material layer 206. FIG. 2B shows a second sensor architecture 210 that comprises a substrate layer 212, a gold or other electrically conductive layer 214 formed on a top surface of the substrate layer 212, and a metallic fractal nanostructure layer 216 formed on a top surface of the gold or other electrically conductive layer 214. FIG. 2C shows a third sensor architecture 218 that comprises a substrate layer 220, a first graphene or other 2D material layer 222 formed on a top surface of the substrate layer 220, a first metallic fractal nano-fractal nanostructure layer 224 from on a top surface of the first graphene or other 2D material layer 222, a second graphene or other 2D material layer 226 formed on a top surface of the first metallic fractal nano-fractal nanostructure layer 224, and a second metallic fractal nano-fractal nanostructure layer 228 formed on a top surface of the second graphene or other 2D material layer 226. FIG. 2D shows a fourth sensor architecture 230 that comprises a substrate layer 232, a first graphene or other 2D material layer 234 formed on a top surface of the substrate layer 232, a first metallic fractal nano-fractal nanostructure layer 236 from on a top surface of the first graphene or other 2D material layer 234, a second graphene or other 2D material layer 238 formed on a top surface of the first metallic fractal nano-fractal nanostructure layer 236, and a first metallic nanodot layer 240 formed on a top surface of the second graphene or other 2D material layer 238.

In some examples, the metallic nanofractals formed on the graphene or other conducting surfaces can include, but are not limited to, other 2D materials such as MoS₂, black phosphorous, WS₂, etc. The sensor architecture can include metallic nanofractals that are formed on a metal coated silicon/glass/quartz wafer, as shown in FIG. 2B. The sensor architecture can include a multilayer architecture in the form of graphene-nanofractals-graphene-nanofractals fabricated by consecutive graphene transfers and electroplating, as shown in FIG. 2C. The sensor architecture can include a multilayer architecture in the form of graphene-nanofractals-graphene-nanodots fabricated by consecutive graphene transfers and electroplating and physical vapor deposition, as shown in FIG. 2D. In some examples, the base layer of the fractal nanostructures can be a flat opaque/transparent substrate (e.g., silicon, germanium, glass, quartz, polymer, gel etc.). Also, the base substrate can be a flexible (Kapton tape, polymer, gel, pdms etc.) or a rigid surface.

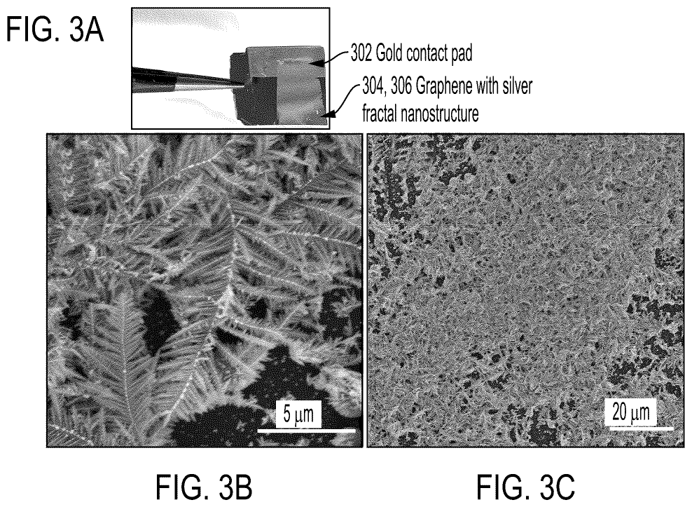

FIG. 3A shows a photograph of a sensor architecture with silver fractal nanostructures electroplated on a single layer graphene with a SiO₂—Si layer as substrate base according to examples of the present disclosure. FIG. 3B and FIG. 3C show closeup and large area scanning electron microscope views, respectively, of the silver fractal nanostructures on graphene according to examples of the present disclosure. The conducting layer can be 2D atomic layer like graphene, MoS₂, WS₂, black phosphorus with or without additional modifications (e.g., doping, functionalization, oxidation, patterning) of the conducting layer, as shown in FIG. 3A. A metallic contact pad (gold, copper, aluminum etc.) may be deposited for better current transport during electroplating. As shown in FIG. 3A, a gold contact pad 302 is deposited on a graphene layer 304 for better current transport through the current leads and graphene ensuring a uniform deposition. The fractal nanostructure layer 306 is deposited on the graphene layer 304. FIG. 3B and FIG. 3C show closeup and large area scanning electron microscope views, respectively, of the silver fractal nanostructures on graphene.

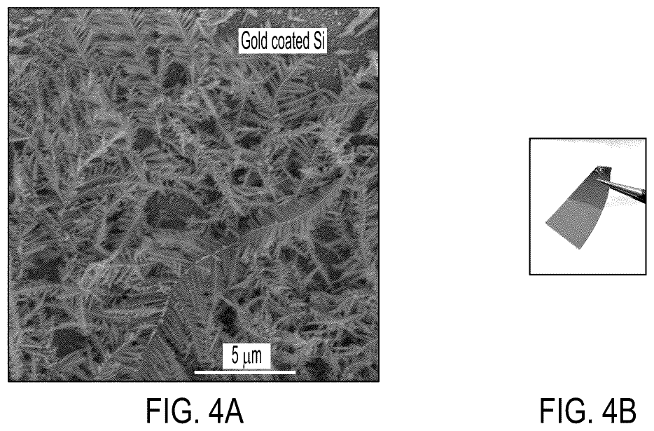

FIG. 4A shows a scanning electron microscope view of the silver fractal nanostructures on gold coated Si according to examples of the present disclosure. FIG. 4B shows a photograph of the example device in the inset of FIG. 4A. The conducting coating can be metals (gold, silver, platinum, copper etc.) or conducting oxides like ITO or doped semiconductors or conducting polymers. The fractal nanostructures can be made of silver, gold, platinum, copper, aluminum etc. or combinations thereof.

The sensors composed of fractal feature dimensions can range from sub nanometer to millimeter scale. The sensors composed of fractal nanostructures can be applied on a variety of micro and macro substrates including swabs, pins, needles, Q tips, fabrics, and surfaces of furniture, walls or as a skin tattoo. The sensors composed of fractal nanostructures can be placed on periodic patterns, microwells or other kinds of regular/irregular patterns. Additional patterning of the conducting layer may be included to enable deposition at specific locations.

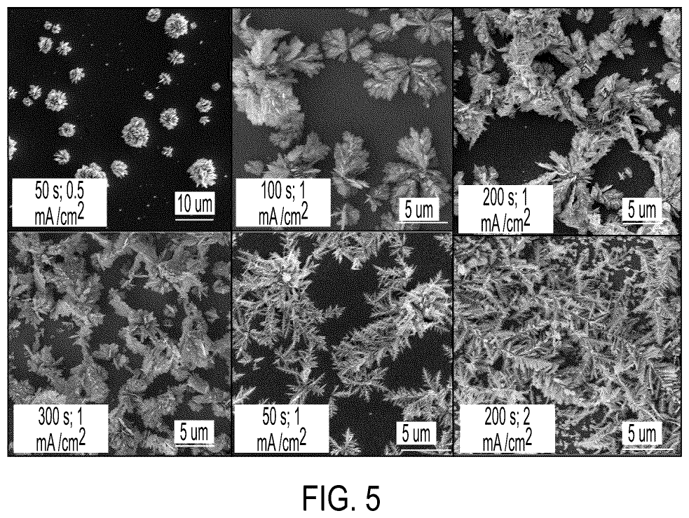

FIG. 5 shows a scanning electron microscope view of the silver fractal nanostructures electrodeposited on ITO coated glass surface with various parameters marked in the figure according to examples of the present disclosure. The electrolyte concentration of silver nitrite is kept constant at 2 mg/ml. The density of the fractal surface may be controlled using different current density (picoampere to ampere/square meter), electrolyte concentration and time of deposition (milliseconds to hours).

The fractal nanostructure can be modified chemically, with DNA or biomolecules like antibody or antigen for greater sensitivity and multiplexed detection. Fluorophores or Raman reporters can be sprinkled or functionalized on the fractal nanostructures for further sensitivity or added functionality. The fractal substrate can be a stand-alone device or combined with a microfluidic device or a lab-on-a chip device for easier sample collection and analysis along with multiplexed detection.

The biosensors composed of the fractal nanostructures can be used for diagnosis of various pathogens, including but are not limited to, viruses, bacteria, and/or fungi. The viruses can include coronaviruses other than SARS-CoV-2 like influenza, Zika, Marburg and other respiratory/non-respiratory viruses. The sensor can also be used for the detection of other pathogens like bacteria, biomolecules and proteins. The pathogen samples can be saliva, swab, blood plasma, urine, tears, or any form of physiological body fluids carrying the live/dead virus/pathogen. The sample can be directly used with/without further processing like some form of chemical or physical treatment.

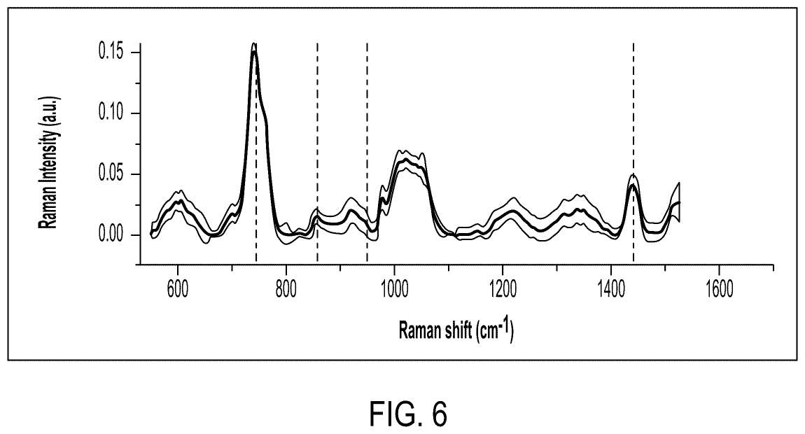

FIG. 6 shows a Raman spectra of cell lysates containing inactivated SARS-CoV-2 acquired on silver fractal nanostructures after post-processing according to examples of the present disclosure. Spectra is recorded using a bench top Raman spectrometer/hand-held spectrometer/portable spectrometer. Preprocessing like background correction, normalization, cosmic ray removal etc. is applied to the collected spectra for consistency.

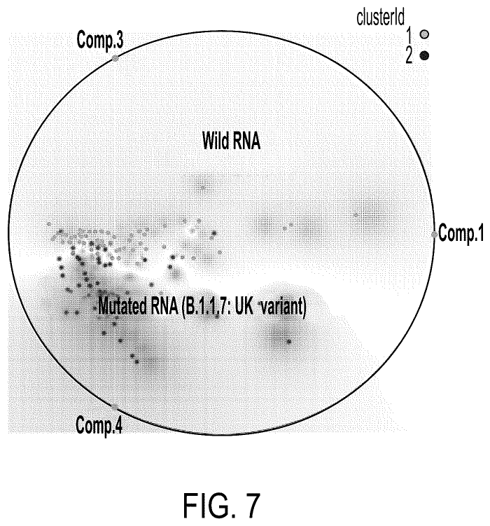

FIG. 7 shows a principal component analysis that show spectral data points belonging to RNA extracted from wild and mutated SARS-CoV-2 cluster together according to examples of the present disclosure. Unsupervised classification algorithm like principal component analysis or K-means clustering is used to classify the data sets from different samples (e.g., RNA extracted from wild type and mutated SARS-CoV-2).

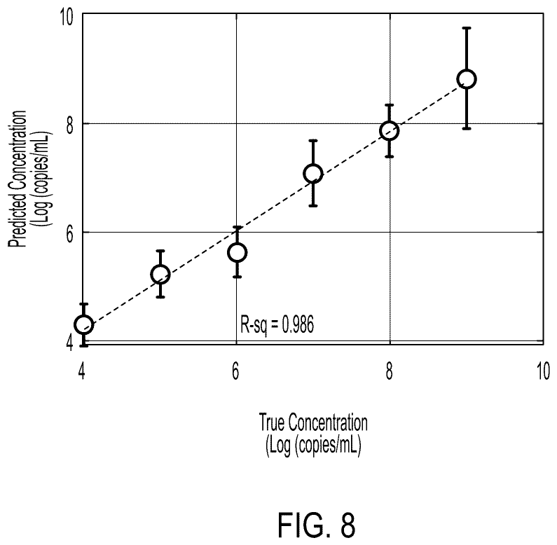

Partial least square analysis with/without additional outlier detection algorithm like robust PCA is applied to the acquired viral dataset on the silver fractal substrate for determination of viral concentration, as shown in FIG. 8. Experimental limit of detection (LOD) is 10⁴ copies/ml (asymptotic range). Theoretic LOD is estimated to be lower. Since the spectral differences are subtle, supervised machine learning algorithms like logistic regression, support vector machine, decision tree, and random forest may be applied to boost the sensor performance.

FIG. 8 shows a predicted concentration versus true concentration of inactivated SARS-CoV— in cell lysates by partial least square regression according to examples of the present disclosure. Experimental LOD is 10⁴ copies/ml. The SERS signal was collected on silver fractal substrate.

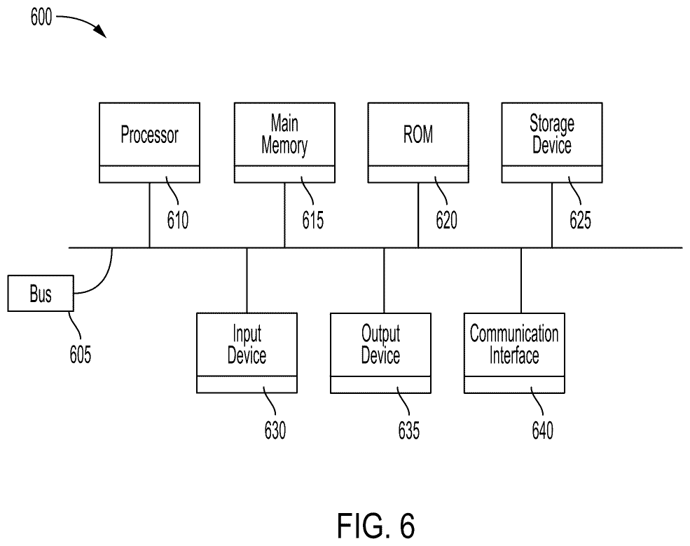

FIG. 9 illustrates example components of a device 900 that may be used within environment described above and comprises one or more components of device 900.

As shown in FIG. 9, device 900 may include a bus 905, a processor 910, a main memory 915, a read only memory (ROM) 920, a storage device 925, an input device 930, an output device 935, and a communication interface 940.

Bus 905 may include a path that permits communication among the components of device 900. Processor 910 may include a processor, a microprocessor, an application specific integrated circuit (ASIC), a field programmable gate array (FPGA), or another type of processor that interprets and executes instructions. Main memory 915 may include a random access memory (RAM) or another type of dynamic storage device that stores information or instructions for execution by processor 910. ROM 920 may include a ROM device or another type of static storage device that stores static information or instructions for use by processor 910. Storage device 925 may include a magnetic storage medium, such as a hard disk drive, or a removable memory, such as a flash memory.

Input device 930 may include a component that permits an operator to input information to device 900, such as a control button, a keyboard, a keypad, or another type of input device. Output device 935 may include a component that outputs information to the operator, such as a light emitting diode (LED), a display, or another type of output device. Communication interface 940 may include any transceiver-like component that enables device 900 to communicate with other devices or networks. In some implementations, communication interface 940 may include a wireless interface, a wired interface, or a combination of a wireless interface and a wired interface. In embodiments, communication interface 940 may receiver computer readable program instructions from a network and may forward the computer readable program instructions for storage in a computer readable storage medium (e.g., storage device 925).

Device 900 may perform certain operations, as described in detail below. Device 900 may perform these operations in response to processor 910 executing software instructions contained in a computer-readable medium, such as main memory 915. A computer-readable medium may be defined as a non-transitory memory device and is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. A memory device may include memory space within a single physical storage device or memory space spread across multiple physical storage devices.

The software instructions may be read into main memory 915 from another computer-readable medium, such as storage device 925, or from another device via communication interface 940. The software instructions contained in main memory 915 may direct processor 910 to perform processes that will be described in greater detail herein. Alternatively, hardwired circuitry may be used in place of or in combination with software instructions to implement processes described herein. Thus, implementations described herein are not limited to any specific combination of hardware circuitry and software.

In some implementations, device 900 may include additional components, fewer components, different components, or differently arranged components than are shown in FIG. 9.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Embodiments of the disclosure may include a system, a method, and/or a computer program product at any possible technical detail level of integration. The computer program product may include a computer readable storage medium (or media) having computer readable program instructions thereon for causing a processor to carry out or execute aspects and/or processes of the present disclosure.

In embodiments, the computer readable program instructions may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on a user's computer, partly on the user's computer, as a stand-alone software package, partly on the user's computer and partly on a remote computer or entirely on the remote computer or server.

In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

In embodiments, a service provider could offer to perform the processes described herein. In this case, the service provider can create, maintain, deploy, support, etc., the computer infrastructure that performs the process steps of the disclosure for one or more customers. These customers may be, for example, any business that uses technology. In return, the service provider can receive payment from the customer(s) under a subscription and/or fee agreement and/or the service provider can receive payment from the sale of advertising content to one or more third parties.

The foregoing description provides illustration and description, but is not intended to be exhaustive or to limit the possible implementations to the precise form disclosed. Modifications and variations are possible in light of the above disclosure or may be acquired from practice of the implementations.

It will be apparent that different examples of the description provided above may be implemented in many different forms of software, firmware, and hardware in the implementations illustrated in the figures. The actual software code or specialized control hardware used to implement these examples is not limiting of the implementations. Thus, the operation and behavior of these examples were described without reference to the specific software code—it being understood that software and control hardware can be designed to implement these examples based on the description herein.

Even though particular combinations of features are recited in the claims and/or disclosed in the specification, these combinations are not intended to limit the disclosure of the possible implementations. In fact, many of these features may be combined in ways not specifically recited in the claims and/or disclosed in the specification. Although each dependent claim listed below may directly depend on only one other claim, the disclosure of the possible implementations includes each dependent claim in combination with every other claim in the claim set.

While the present disclosure has been disclosed with respect to a limited number of embodiments, those skilled in the art, having the benefit of this disclosure, will appreciate numerous modifications and variations there from. It is intended that the appended claims cover such modifications and variations as fall within the true spirit and scope of the disclosure.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 5. In certain cases, the numerical values as stated for the parameter can take on negative values. In this case, the example value of range stated as “less than 10” can assume negative values, e.g. −1, −2, −3, −10, −20, −30, etc.

While the invention has been illustrated respect to one or more implementations, alterations and/or modifications can be made to the illustrated examples without departing from the spirit and scope of the appended claims. In addition, while a particular feature of the invention may have been disclosed with respect to only one of several implementations, such feature may be combined with one or more other features of the other implementations as may be desired and advantageous for any given or particular function.

Furthermore, to the extent that the terms “including”, “includes”, “having”, “has”, “with”, or variants thereof are used in either the detailed description and the claims, such terms are intended to be inclusive in a manner similar to the term “comprising.” As used herein, the phrase “one or more of”, for example, A, B, and C means any of the following: either A, B, or C alone; or combinations of two, such as A and B, B and C, and A and C; or combinations of three A, B and C.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A pathogen detection system comprising:
a pathogen sensor comprising a plasmonically active pathogen sensing area that comprises a hybrid structure of electrically conductive surface and metal nanofractals, forming a surface enhanced Raman spectroscopic (SERS) substrate; and
a controller comprising a memory storing one or more trained machine learning algorithms, that perform label-free identification and differentiation of a pathogen or the pathogen and a pathogen mutation based on acquired Raman spectra.

2. The pathogen detection system of claim 1, wherein the controller is further configured to determine one or more of the following based on the one or more trained machine learning algorithms and the acquired Raman spectra: pathogen concentrations, or differences in various protein and genomic compositions of the pathogen or the pathogen and the pathogen mutation.

3. The pathogen detection system of claim 1, wherein the plasmonically active pathogen sensing area is fabricated using metal nano fractals on an electrically conducting surface, through a process of electrodeposition.

4. The pathogen detection system of claim 3, wherein the metal nano fractals comprise silver, gold, platinum, copper, aluminum, or any combination/alloy of these metals.

5. The pathogen detection system of claim 3, wherein the electrically conducting surface is a 2D material, wherein the 2D material comprises graphene, MoS₂, black phosphorus, WS₂ or other 2D electrically conductive materials.

6. The pathogen detection system of claim 5, wherein the 2D material is modified or unmodified, wherein the modified comprises doping, functionalization, oxidation, or patterning of the electrically conductive surface.

7. The pathogen detection system of claim 3, wherein the SERS substrate comprises a metal coated silicon wafer, a metal coated glass wafer, or a metal coated quartz wafer.

8. The pathogen detection system of claim 3, wherein the electrically conductive surface for the SERS substrate comprises a metal, a conducting oxide, a doped semiconductor, or a conducting polymer, wherein the metal comprises gold, silver, platinum, or copper, the conducting oxide comprises ITO, zinc oxide, gallium oxide, indium oxide or tin oxide.

9. The pathogen detection system of claim 1, wherein the plasmonically active pathogen sensing area comprises a multilayered architecture in the form of graphene-nanofractals-graphene-nanofractals fabricated by consecutive graphene transfers and electrodeposition.

10. The pathogen detection system of claim 1, wherein the plasmonically active pathogen sensing area comprises a multilayered architecture in the form of graphene-nanofractals-graphene-nanodots fabricated by consecutive graphene transfers and electroplating, physical vapor deposition, chemical synthesis, or combinations thereof.

11. The pathogen detection system of claim 1, wherein the plasmonically active pathogen sensing area comprises a substrate, a first graphene or other 2D material layer formed on a top surface of the substrate, and a first metallic fractal nanostructure formed on a top surface of the first graphene or other 2D material layer.

12. The pathogen detection system of claim 11, wherein the plasmonically active pathogen sensing area further comprises a second graphene or other 2D material layer formed on the top surface of the first metallic fractal nanostructure and a second metallic fractal nanostructure formed on a top surface of the second graphene or other 2D material layer.

13. The pathogen detection system of claim 11, wherein the plasmonically active pathogen sensing area further comprises a second graphene or other 2D material layer formed on the top surface of the first metallic fractal nanostructure and a plurality of metallic nanodots formed on a top surface of the second graphene or other 2D material layer.

14. The pathogen detection system of claim 11, wherein the substrate comprises a flat transparent material or a flat opaque material, the flat transparent material or a flat opaque material comprising silicon, germanium, glass, quartz, polymer, or a gel.

15. The pathogen detection system of claim 1, wherein the SERS substrate has a dimension ranging from about a micron to about a meter.

16. The pathogen detection system of claim 1, wherein the metal nanofractals have a dimension ranging from about a sub-nanometer to about a millimeter.

17. The pathogen detection system of claim 1, wherein the pathogen sensor is applied to a micro or macro substrate, wherein the micro or macro substrate comprises a flute that operates as a breath sensor, a swab, a pin, a needle, a cotton swab, a fabric, a surface of a furniture, a surface of a wall, or a skin tattoo.

18. The pathogen detection system of claim 1, wherein the pathogen sensor is arranged on a period pattern, a microwell, or other kinds of regular or irregular patterns.

19. The pathogen detection system of claim 1, wherein the electrically conductive surface is patterned to enable deposition of the metal nanofractals at specific locations.

20. The pathogen detection system of claim 1, wherein density or coverage of the metal nanofractals is controlled by using different current densities ranging from about a picoampere per square meter to about an ampere per square meter, different electrolyte concentrations and time of deposition ranging from about a few milliseconds to about a few hours, or both.

21. The pathogen detection system of claim 1, wherein the metal nanofractals are modified chemically with DNA or biomolecules for improved sensitivity and multiplexed detection, wherein the biomolecules comprise an antibody or an antigen.

22. The pathogen detection system of claim 1, wherein the metal nanofractals comprise fluorophores or Raman reporters that are sprinkled or functionalized to improve sensitivity or added functionality.

23. The pathogen detection system of claim 1, wherein the pathogen sensor is a stand-alone device, combined with a microfluidic device, or combined with a lab-on-chip device for easier sample collection and analysis along with multiplexed detection.

24. The pathogen detection system of claim 1, wherein the pathogen comprises a virus, a bacterium, a biomolecule, or a protein.

25. The pathogen detection system of claim 1, wherein the label-free identification and differentiation is based on a specimen acquired by saliva, a swab, blood plasma, urine, tears, or any form of physiological body fluids carrying live or dead pathogen.

26. The pathogen detection system of claim 25, wherein the specimen is collected with or without further processing comprising a chemical or physical treatment.

27. The pathogen detection system of claim 25, wherein the specimen is collected by directly drop casting, placing the specimen on the pathogen sensor, blowing, sneezing, or coughing directly on the pathogen sensor.

28. The pathogen detection system of claim 1, wherein the one or more machine-learning algorithms are trained by dividing collected datasets into a training dataset, a validation dataset, and a test dataset.

29. The pathogen detection system of claim 1, wherein the one or more machine-leaning algorithms comprise an unsupervised classification algorithm that performs a data visualization operation and a feature extraction operation, wherein the unsupervised classification algorithm comprises principal component analysis, K-means clustering, or other visualization algorithms.

30. The pathogen detection system of claim 1, wherein the one or more machine-learning algorithms comprise a partial least squares analysis with or without additional outliner detection algorithm applied to an acquired pathogen dataset on the SERS substrate for determining of pathogen concentration of at least 10 copies/ml or higher.

31. The pathogen detection system of claim 30, wherein the additional outliner detection algorithm comprises a robust principal component analysis (PCA).

32. The pathogen detection system of claim 1, wherein the one or more machine-learning algorithms comprise one or more supervised machine learning algorithms, wherein the one or more supervised machine learning algorithms comprise a logistic regression, a support vector machine, a decision tree, a random forest that is used to identify one or more types of pathogens in a pathogen specimen and to differentiate between various mutations.

33. A method for label-free detection to identify and differentiate various pathogens and their mutations based on applying a machine learning algorithm on surface enhanced Raman spectra acquired on a nano fractal substrate, the method comprising:
depositing a pathogen specimen onto a pathogen sensor comprising plasmonically active noble metal nanostructures;
illuminating the pathogen specimen that is deposited with a laser;
acquiring surface enhanced Raman spectroscopic (SERS) signal from the pathogen specimen; and
determining and identifying one or more pathogens and their mutations by applying the machine learning algorithm on the SERS signal that is acquired.

34. A method of forming a pathogen sensor, the method comprising:
forming a first graphene or a 2D material layer on a top surface of a substrate; and
forming a first metallic fractal nanostructure on a top surface of the first graphene or a 2D material layer.

35. The method of forming a pathogen sensor of claim 34, further comprising forming a second graphene or a 2D material layer on the top surface of the first metallic fractal nanostructure and forming a second metallic fractal nanostructure on a top surface of the second graphene or a 2D material layer.

36. The method of forming a pathogen sensor of claim 35, further comprising forming a second graphene or a 2D material layer on the top surface of the first metallic fractal nanostructure and forming of layer of a plurality of metallic nanodots on a top surface of the second graphene or a 2D material layer.

37. The method of forming a pathogen sensor of claim 36, wherein the substrate comprises a flexible material or a rigid material, wherein the flexible material comprises, a Kapton tape, a polyimide film, a polymer, a gel, or a polydimethylsiloxane (PDMS).

38. A method of forming a pathogen sensor, the method comprising:
forming an electrically conductive layer formed on a top surface of a substrate; and
forming a metallic fractal nanostructure formed on a top surface of the electrically conductive layer.
